(19) United States
(12) Patent Application Publication
(10) Pub. No.: US 2012/0034314 A1
(43) Pub. Date: Feb. 9, 2012

(54) Antiseptic Liquid Formulation, A Method for Its Use, and A Method for Preparing the Formulation

(76) Inventors: Lisa Turner Levison, St. George Island, FL (US); Christine Anne Forshee, Marietta, GA (US); Kerry Hays Gronewald, Atlanta, GA (US)

(21) Appl. No.: 12/851,154

(22) Filed: Aug. 5, 2010

(51) Int. Cl. A01N 59/16; A61Q 19/10; A61K 8/40; A01N 59/20

(52) U.S. Cl. 424/618; 424/638; 510/131

(57) ABSTRACT

An antiseptic formulation capable of providing antimicrobial properties over an extended period of time is disclosed. The formulation includes chelated metal ions and a fixative polymer with the capacity to bond the chelated metal ions to the skin. This fixative polymer, polyquaternium-69 has the ability to bond chelated metal ions, and other types of antimicrobial agents, to the skin for periods of hours or days depending on the conditions in which it is used. An antiseptic liquid formulation can be integrated in a soap product and a spray product. An antiseptic gel formulation is further disclosed.

### FIELD OF THE INVENTION

The present antiseptic liquid formulation relates to long-lasting antimicrobial solution, which can comprise one or more antimicrobial agents, which can be bonded to skin using one or more polymers, thus providing antimicrobial protection over an extended period of time.

### BACKGROUND

Antiseptic liquid formulations designed to kill germs on the skin have existed for over a century. For example, the antiseptic properties of various alcohols have been known since the mid-nineteenth century. Antiseptic, or antibacterial soaps have existed for many decades and are commonly used to prevent the spread of infectious diseases, which are often transmitted on skin, particularly that of the hands. Such soaps typically contain antimicrobial compounds such as triclosan, which is a polychlorinated phenoxyphenol capable of killing a broad spectrum of potentially harmful microbes such as viruses, bacteria and fungi.

More recently, liquid hand sanitizers have become more common. These products often contain ethyl alcohol, which has two important characteristics. Namely, it is a highly effective antimicrobial agent and it dries very quickly, allowing the user to sanitize his or her hands without water, soap or towels. The antimicrobial effect of both antibacterial soap and liquid hand sanitizers is not long lasting and the user's hands quickly become recontaminated.

Some hand sanitizers also contain triclosan, which can remain on the skin for a few hours after application because it is not washed off as it would be when used in antibacterial soap. See U.S. Patent Application Publication No. US 2003/0008791. However, this extended activity has not been shown to last more than four to six hours, and often does not persist for more than a couple of hours depending on the conditions in which it is used.

Chelated metal ions, particularly those comprising ionized silver and copper, have been shown to exhibit long lasting antimicrobial activity. For example, U.S. Pat. No. 7,311,927 to Miner et al. discloses an antiseptic solution comprising silver ion chelated with polypectate and ethylenediaminetetraacetic acid (EDTA), which has shown significant antimicrobial activity for up to twenty-one (21) hours. These chelated compounds have not only proven to be effective killers of disease-causing bacteria and viruses, but have proven invulnerable to the problem of antibiotic resistance, wherein, over time bacteria develop a resistance against the antimicrobial activity of a particular agent. However, these chelated compounds, like any other antimicrobial agent, can only remain effective on the skin if they can remain there for an extended period.

What is needed is an antiseptic liquid formulation that combines one or more long-lasting antimicrobial agents with a polymer that can allow such antimicrobial agents to adhere to the surface of the skin for an extended period of time.

### SUMMARY

The present antiseptic liquid formulation utilizes a fixating polymer to bond chelated metals and other antimicrobial agents to skin for an extended period of time, providing long-lasting protection against bacteria, viruses and other harmful microbes.

In a first embodiment, the present antiseptic liquid formulation can comprise a chelated metal ion bond bonded to the skin by a solution further comprising the fixing polymer, polyquaternium-69, ethyl alcohol and water.

In a second embodiment, the present antiseptic liquid can be used in a hand-sanitizing gel formulation comprising long-lasting antimicrobial compounds, namely chelated silver ions and triclosan, encapsulated in polyquaternium-69. The liquid antiseptic gel can comprise: ethyl alcohol; water; glycereth-18; polyoxyethylene (20) sorbitan monooleate; 2- amino-2-methyl-1-propanol; acrylate crosspolymer; polyquaternium-69; citric acid and sodium citrate; and Irgasan DP 300 USP.

In a third embodiment, the present antiseptic liquid can be used in a hand-sanitizing spray formulation comprising: ethyl alcohol; water; glycereth-18; polyoxyethylene (20) sorbitan monooleate; p2-amino-2-methyl-1-propanol; polyquaternium-69; citric acid and sodium citrate; and Irgasan DP 300 USP.

In a fourth embodiment, the present antiseptic liquid formulation can be used in an antibacterial soap. This antibacterial soap can comprise: water; sodium laureth sulfate; sodium lauryl sulfate; hydrolyzed jojoba esters; propylene glycol; cocamidopropyl betaine; ethyl alcohol; cocamide DEA, macadamia glycerides; acrylate crosspolymer; polyquaternium-69; phenoxyethanol; silver dihydrogen citrate; and Irgasan DP 300 USP and tetrasodium EDTA.

### BRIEF DESCRIPTION OF THE DRAWINGS

An antiseptic liquid formulation, a method for preparing the formulation, and a method of using the formulation can be better understood with reference to the following figures. The components within the figures are not necessarily to scale, emphasis instead being placed upon clearly illustrating the principles of suspending antimicrobial compounds within a polymer that bonds the antimicrobial compounds to the skin for an extended time. Moreover, in the figures, like reference numerals designate corresponding parts throughout the different views.

FIG. 1 is a flow diagram illustrating an embodiment of a method for preparing an antiseptic liquid formulation.

FIG. 2 is a flow diagram illustrating an embodiment of a method for using the antiseptic liquid formulation of FIG. 1.

### DETAILED DESCRIPTION

An antiseptic liquid formulation can comprise one or more long-lasting antimicrobial agents, including, but not limited to ethyl alcohol, triclosan, or copper or silver chelates or ions, thymol, chlorhexidine gluconate, quaternary ammonium compounds such as benzalkonium chloride, cetyl trimethylammonium bromide, cetylpyridinium chloride, and benzethonium chloride. In a preferred embodiment, chelated silver ion, in the form of silver acrylate can be used to provide long-lasting antimicrobial activity. Silver acrylate can be dispersed in a polymer, which may then be applied to the skin. A polymer, which has shown the capacity to disperse silver acrylate and other antimicrobial agents, as well as to form a bond with the skin is the fixative polymer, polyquaternium-69, which was originally designed for use in hair styling products. Polyquaternium-69, known commercially as Aquastyle 300™, is a tetrapolymer of vinyl caprolactam (VCL), vinylpyrrolidone (VP), dimethylaminopropyl methacrylamide (DMAPMA) and C₉-C₂₄ alkyl quaternized dimethylaminopropyl methacrylic acid or quaternized (meth) acrylamide monomers, described in U.S. Pat. No. 6,852,815, which is incorporated herein in its entirety.

When polyquaternium-69 is combined with antimicrobial agents, including chelated silver, and other surfactants and solvents, a liquid that is fast drying, with little or no tackiness can be achieved, which can bond various antimicrobial agents to the skin for several days. Furthermore, this bond has been shown not to adversely affect the antimicrobial agents' ability to kill microbes, which come into contact with the skin, but to allow the antimicrobial agents to continue killing microbes. The present antiseptic liquid formulation has been adjusted for use in hand sanitizing gels, hand-sanitizing sprays, and antibacterial soaps, but can also be used in any number of other skincare products, such as make-ups, lipbalms, hand lotions, total body moisturizers, massage lotions and body washes.

The present antiseptic liquid formulation can be produced by mixing a first solution of ethyl alcohol and water in a first container. Irgasan DP 300 USP, more commonly known as triclosan, can be dissolved in this first solution. The first solution can be completed by adding gelling agents, surfactants and a water soluble and alcohol soluble ester that acts as a glycerin substitute (Glycereth-18) to the first container. A second solution can be produced by mixing silver dihydrogen citrate with a base comprised of an acrylate crosspolymer, such as Ultrarez 21, and water, creating silver acrylate in a second container. The first solution and second solution can then be combined to form the present antiseptic liquid formulation, which can comprise three antimicrobial agents, namely ethyl alcohol, triclosan and silver acrylate. The pH of the combined solutions can then be adjusted to somewhere between 6 and 9. Polyquaternium-69 can be diluted in ethyl alcohol and added to the combined first and second solution. The antiseptic liquid formulation created is not hazy or tacky and does not bead as it dries on the skin. The formula creates a breathable bond that allows the hand to perspire normally. Normal perspiration, excretion of the skin and contact with water may affect the lasting power of the bond, but the triclosan and silver acrylate will remain on the skin in traces powerful enough to continue killing microbes.

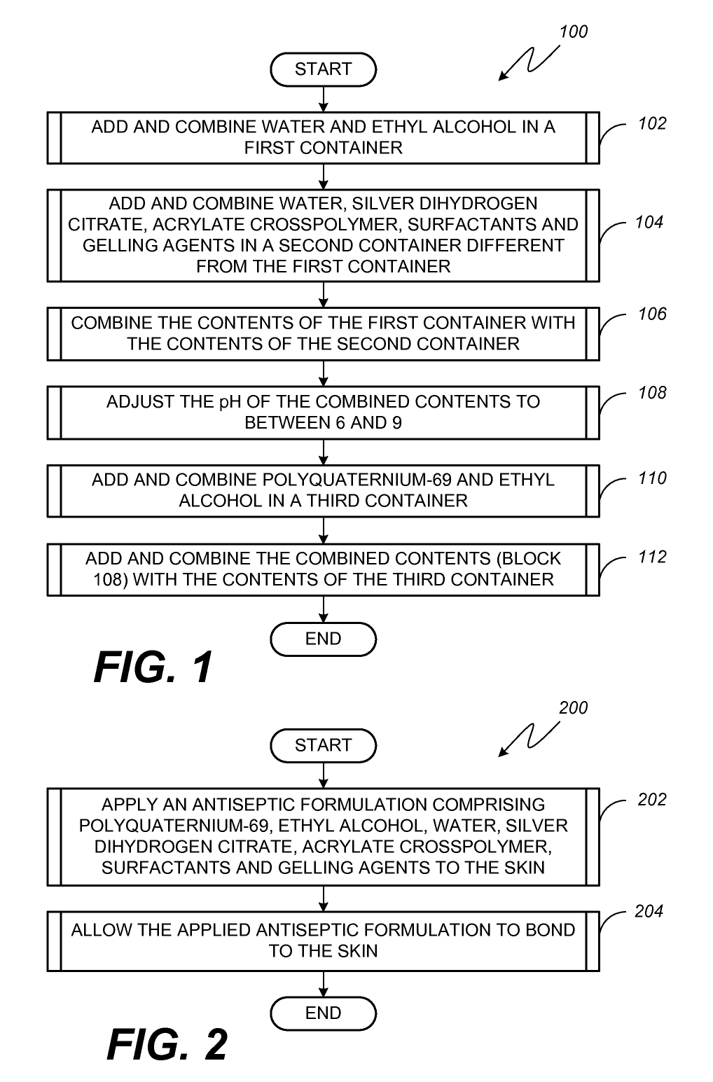

FIG. 1 illustrates an embodiment of a method 100 for preparing the antiseptic liquid formulation. In block 102, water and ethyl alcohol are added and combined in a first container. In block 104, water, silver dihydrogen citrate, acrylate crosspolymer, surfactants and gelling agents are added and combined in a second container different from the first container. The above-described functions associated with block 102 and 104 can be performed in the illustrated sequence, the reverse sequence or substantially simultaneously with the other. However, the components of the first container and the second container are combined within the respective containers, the method 100 continues with block 106 where the contents of the first container are combined with the contents of the second container. Thereafter, in block 108, the pH of the combined contents is adjusted to between 6 and 9. In block 110, polyquaternium-69 and ethyl alcohol are introduced and combined in a third container such that the polyquaternium-69 is dissolved in the ethyl alcohol. In block 112, the combined contents from the first and second containers is combined with the contents of the third container.

FIG. 2 illustrates an embodiment of a method 200 for using the antiseptic liquid formulation. In block 202, an antiseptic formulation comprising polyquaternium-69, ethyl alcohol, water, silver dihydrogen citrate, acrylate crosspolymer, surfactants and gelling agents is applied to the skin. As described, the antiseptic formulation can be in any of a liquid soap, spray or gel configurations. In block 204, the applied antiseptic formulation bonds to the skin. As also described, the antiseptic formulation bonds to the skin as it dries on the skin.

When this antiseptic liquid formulation is applied to the skin, all three antimicrobial agents immediately begin actively killing microbes on contact. The ethyl alcohol will then quickly evaporates leaving both triclosan and silver acrylate bonded to the skin by polyquaternium-69 and continuing to work as active antimicrobial agents. Triclosan can continue to be an active antimicrobial in the medium term and silver acrylate can continue to be an active antimicrobial in the long term. Heat and humidity do not substantially affect the present antiseptic formulation as polyquaternium-69 was designed to withstand heat and especially high humidity. Eventually, the formulation's effectiveness terminates due to excretion of the skin over a period of days.

Table I lists the components comprising a hand-sanitizing gel formulation, and their preferred amounts and acceptable ranges by weight percent.

TABLE I
Hand-Sanitizing Gel Formulation

| | Acceptable | Preferred |
|---|---|---|
| Component | Range (Wt. %) | Amount (Wt. %) |
| Ethyl Alcohol (190 proof) | 58-95 | 58 |
| Water (deionized) | 7-40 | 29.1 |
| Glycereth-18 (Hest G-18-0) | 2-7 | 5 |
| Polyoxyethylene (20) Sorbitan | 2-6 | 5 |
| Monooleate (Polysorbate 80) | | |
| 2-Amino-2-methyl-1-propanol | 0.1-2 | 1 |
| (AMP-95) | | |
| Acrylate Crosspolymer | 0.1-2 | 0.8 |
| (Ultrarez 21) | | |
| Polyquaternium-69 | 0.5-1 | 0.5 |
| (Aquastyle 300) | | |
| Silver Dihydrogen Citrate | 0.1-0.9 | 0.3 |
| (Tinosan SDC-R) | | |
| Irgasan DP 300 USP (Triclosan) | 0.3-2 | 0.3 |

Traditional hand sanitizers have no residual activity. In less than 60 seconds, they leave you completely unprotected from dangerous germs. The present hand-sanitizing gel formulation kills germs instantly on contact, but continues to prevent cross contamination of surfaces contacted by the hands for hours. It is also infused with a pharmaceutical-grade moisturizer, Glycereth-18, that counteracts the drying effects of alcohol. This moisturizer also helps to protect the user from harmful microbes by working with the body's own oils, to restore the skin's natural defenses, which can be compromised by the drying effects of alcohol.

The present hand-sanitizing gel's unique ability to kill germs has been demonstrated in laboratory test results where zone of inhibition tests were performed. Under normal circumstances where zone of inhibition tests are performed on antiseptics, antibiotics or traditional hand sanitizers, the zone is usually somewhere between 1 and 4 millimeters. In some cases, the zone of inhibition in derma-glove test results reached up to 21 millimeters. Furthermore, zone of inhibition tests performed on traditional, alcohol-based hand sanitizers are carried out by inoculating the bacteria only once, usually after about 60 seconds on the agar plate. In zone of inhibition testing for the present hand-sanitizing gel, the bacteria was inoculated at 60 seconds, 30 minutes, 1 hour, 2 hours, 4 hours, 8 hours, 24 hours and 48 hours and still, the zone remained to at least 9 millimeters.

In the present test, 4.5 grams of the present hand-sanitizing gel formulation was distributed over 100 square inches of a plastic, sold commercially as Whirl-Pak™. One inch squares of the treated plastic were placed on plates inoculated with active pseudomonas cultures. The present hand-sanitizing gel formulation was dried at room temperature for one minute before testing was started.

Table II lists the zone of inhibition test results for the present hand-sanitizing gel formulation using pseudomonas bacterium.

TABLE II
Zone of Inhibition Test Results for the Hand-Sanitizing
Gel Formulation

| Hours | Millimeters |
|---|---|
| 0 | 21 |
| 0.5 | 21 |
| 1 | 20 |
| 4 | 20 |
| 8 | 19 |
| 16 | 17 |
| 24 | 12 |
| 48 | 9 |

Table III lists the components comprising a hand-sanitizing spray formulation, and their preferred amounts and acceptable ranges by weight percent. This spray requires a much thinner, less viscous liquid in order to comply with the demands of spray bottle delivery systems.

TABLE III
Hand-Sanitizing Spray Formulation

| | Acceptable | Preferred |
|---|---|---|
| Component | Range (Wt. %) | Amount (Wt. %) |
| Ethyl Alcohol (190 proof) | 58-95 | 58 |
| Water (deionized) | 7-40 | 29.9 |
| Glycereth-18 (Hest G-18-0) | 1-6 | 5 |
| Polyoxyethylene (20) Sorbitan | 1-6 | 5 |
| Monooleate (Polysorbate 80) | | |
| Polyquaternium-69 (Aquastyle 300) | 0.5-1 | 0.5 |
| Silver Dihydrogen Citrate (Tinosan | 0.1-0.9 | 0.3 |
| SDC | | |
| Irgasan DP 300 USP (Triclosan) | 0.3-2 | 0.3 |

Due to the fact that antiseptic spray applications require a much thinner, less viscous liquid in order to exit a spray top, they often contain high amounts of water and alcohol and evaporate much more quickly than gel formulations. Although the present hand-sanitizing spray formulation is a thinner and less viscous formulation, the blending process still allows for an infusion of the fixative polymer wherein the triclosan and silver acrylate can be encapsulated within the polyquaternium-69, so the spray also features residual activity against germs even after drying.

Table IV lists the components comprising an antibacterial soap formulation and their preferred amounts and acceptable ranges by weight percent. In addition to the antiseptic liquid formulation, this antibacterial soap also contains surfactants and moisturizers commonly found in commercially available antibacterial soaps.

TABLE IV
Antibacterial Soap Formulation

| | Acceptable | Preferred |
|---|---|---|
| Component | Range (Wt. %) | Amount (Wt. %) |
| Water (deionized) | 25-36 | 29.8 |
| Sodium Laureth Sulfate | 25-35 | 28 |
| (SLES-2 Colonial) | | |
| Sodium Lauryl Sulphate | 10-20 | 14.5 |
| (Sulfochem ES-2) | | |
| Hydrolyzed Jojoba Esters) | 0.1-6 | 4.4 |
| Floroesters K-20W Jojoba | | |
| Propylene Glycol | 1-5 | 3 |
| (Propylene Glycol FG) | | |
| Cocamidopropyl Betaine | 1-5 | 3 |
| (Lexaine C) | | |
| Ethyl Alcohol | 1-10 | 3 |
| Sodium Laureth Sulphate | 1-5 | 2.6 |
| (Coladet Nana) | | |
| Cocamide DEA | 2-3 | 2.2 |
| Macadamia Glycerides | 1-1.9 | 1.75 |
| (Florosolves Peg16 Macadamia) | | |
| Acrylate Crosspolymer (Ultrarez 21) | 0.5-10 | 6 |
| Polyquaternium-69 (Aquastyle 300) | 0.2-1 | 0.5 |
| Phenoxyethanol (Uniphen P-23) | 0.1-0.5 | 0.4 |
| Silver Dihydrogen Citrate | 0.1-0.9 | 0.3 |
| (Tinosan SDC) | | |
| Irgasan DP 300 USP (Triclosan) | 0.3-2 | 0.3 |
| Tetrasodium EDTA (Versene Na2) | 0.1-0.5 | 0.25 |

Although the invention has been described in terms of exemplary embodiments, it is not limited thereto. Rather, the appended claims should be construed broadly, to include other variants and embodiments of the invention, which may be made by those skilled in the art without departing from the scope and range of equivalents of the invention.

What is claimed is:

1. An antiseptic liquid formulation comprising:
polyquaternium-69;
a sufficient amount of ethyl alcohol required to dissolve said polyquaternium-69;
chelated metal ions; and
water.

2. The antiseptic liquid formulation of claim 1, wherein the chelated metal ion is silver ion.

3. The antiseptic liquid formulation of claim 1, wherein the chelated metal ion is copper ion.

4. The antiseptic liquid formulation of claim 1, further comprising a surfactant.

5. The antiseptic liquid formulation of claim 1, further comprising a gelling agent.

6. The antiseptic liquid formulation of claim 1, further comprising Irgasan DP 300 USP.

7. The antiseptic liquid formulation of claim 1, further comprising thymol.

8. The antiseptic liquid formulation of claim 1, further comprising chlorhexidine gluconate.

9. The antiseptic liquid formulation of claim 1, further comprising quaternary ammonium compounds such as benzalkonium chloride.

10. The antiseptic liquid formulation of claim 1, further comprising cetyl trimethylammonium bromide.

11. The antiseptic liquid formulation of claim 1, further comprising cetylpyridinium chloride.

12. The antiseptic liquid formulation of claim 1, further comprising benzethonium chloride.

13. A method for preparing an antiseptic liquid formulation, comprising the steps of:
adding and combining water and ethyl alcohol in a first container;
adding and combining water, silver dihydrogen citrate, acrylate crosspolymer, surfactants and gelling agents in a second container;
adding and combining the contents of the first container with the second container;
adjusting the pH to between 6 and 9;
adding and combining ethyl alcohol and polyquaternium-69 in a third container; and
adding and combining the contents of the third container and the previously combined contents of the first and second containers.

14. The method of claim 13, which further comprises the step of adding and combining Irgasan DP 300 USP to the water and ethyl alcohol in a first container.

15. The method of claim 13, which further comprises the step of adding and combining thymol to the water and ethyl alcohol in a first container.

16. The method of claim 13, which further comprises the step of adding and combining chlorhexidine gluconate to the water and ethyl alcohol in a first container.

17. The method of claim 13, which further comprises the step of adding and combining quaternary ammonium compounds such as benzalkonium chloride to the water and ethyl alcohol in the first container.

18. The method of claim 13, which further comprises the step of adding and combining cetyl trimethylammonium bromide to the water and ethyl alcohol in the first container.

19. The method of claim 13, which further comprises the step of adding and combining cetylpyridinium chloride to the water and ethyl alcohol in the first container.

20. The method of claim 13, which further comprises the step of adding and combining benzethonium chloride to the water and ethyl alcohol in the first container.

21. A method for using an antiseptic liquid formulation, comprising the steps of:
applying an antiseptic liquid formulation to the skin comprising polyquaternium-69, ethyl alcohol, water, silver dihydrogen citrate, acrylate crosspolymer, surfactants and gelling agents; and
allowing the antiseptic liquid formulation time to bond to the skin.

22. A hand-sanitizing gel formulation comprising:
58-95% ethyl alcohol by weight;
7-40% water by weight;
2-7% glycereth-18 by weight;
2-6% polyoxyethylene (20) sorbitan monooleate by weight;
0.1-2% 2-amino-2-methyl-1-propanol by weight;
0.1-2% acrylate crosspolymer by weight;
0.5-1% polyquaternium-69 by weight;
0.1-0.9% silver dihydrogen citrate by weight; and
0.3-2% Irgasan DP 300 USP by weight.

23. A hand-sanitizing spray formulation comprising:
58-95% ethyl alcohol by weight;
7-40% water by weight;
1-6% Glycereth-18 by weight;
1-6% Polyoxyethylene (20) Sorbitan Monooleate by weight;
0.5-1% Polyquaternium-69 by weight;
0.1-0.9% Silver dihydrogen citrate by weight; and
0.3-2% Irgasan DP 300 USP by weight.

24. An antibacterial soap formulation comprising:
25-36% water by weight;
25-35% sodium laureth sulfate by weight;
10-20% sodium laurel sulfate by weight;
0.1-6% hydrolyzed jojoba esters by weight;
1-5% propylene glycol by weight;
1-5% cocamidopropyl betaine by weight;
2-4% ethyl alcohol by weight;
1-5% sodium laureth sulfate by weight;
2-3% cocamide DEA by weight;
1-1.9% macadamia glycerides by weight;
0.5-10% acrylate crosspolymer by weight;
0.2-1% polyquaternium-69 by weight;
0.1-0.5% phenoxyethanol by weight;
0.1-0.9% silver dihydrogen citrate by weight;
0.3-2% Irgasan DP 300 USP by weight; and
0.1-0.5% tetrasodium EDTA by weight.
